(19) United States
(12) Patent Application Publication
(10) Pub. No.: US 2008/0091026 A1
(43) Pub. Date: Apr. 17, 2008

(54) NOVEL CRYSTAL OF N-[[(5S)-3-[4-(2,6-DIHYDRO-2-METHYLPYRROLO[3,4-C]PYRAZOL-5(4H)-YL)-3-FLUOROPHENYL]-2-OXO-5-OXAZOLIDINYL]METHYL]ACETAMIDE

(75) Inventors: Renato A. Chiarella, Cambridge, MA (US); Matthew Peterson, Hopkinton, MA (US); Lisa Scoppettuolo, Allston, MA (US)

Correspondence Address: PHILIP S. JOHNSON;JOHNSON & JOHNSON, ONE JOHNSON & JOHNSON PLAZA, NEW BRUNSWICK, NJ, 08933-7003, US

(73) Assignee: TRANSFORM PHARMACEUTICALS, INC., Lexington, MA (US)

(21) Appl. No.: 11/869,784

(22) Filed: Oct. 10, 2007

Related U.S. Application Data
(60) Provisional application No. 60/829,056, filed on Oct. 11, 2006.

(51) Int. Cl. C07D 413/14

(52) U.S. Cl. 548/229

(57) ABSTRACT

The present invention relates to a novel crystal of the Active Pharmaceutical Ingredient (API) N-[[(5S)-3 -[4-(2,6-dihydro-2-methylpyrrolo[3,4-c]pyrazol-5(4H)-yl)-3-fluorophenyl]-2-oxo-5-oxazolidinyl]methyl]acetamide, methods for the preparation of this crystal, pharmaceutical compositions comprising this crystal, and methods of treating a patient with this crystal.

### TECHNICAL FIELD

The present invention relates to a novel crystal of the Active Pharmaceutical Ingredient (API) N-[[(5S)-3-[4-(2,6-dihydro-2-methylpyrrolo[3,4-c]pyrazol-5(4H)-yl)-3-fluorophenyl]-2-oxo-5-oxazolidinyl]methyl]acetamide, methods for the preparation of this crystal, pharmaceutical compositions comprising this crystal, and methods of treating a patient with this crystal.

### BACKGROUND OF THE INVENTION

Oxazolidones are a class of synthetic antimicrobial compounds which possess activity against a variety of pathogens. Because of the increasing development of bacterial resistance to many antibiotics, oxazolidones will play an important role in the treatment of infections. One particular oxazolidone which has shown effectiveness in treating infections is N-[[(5S)-3-[4-(2,6-dihydro-2-methylpyrrolo[3,4-c]pyrazol-5(4H)-yl)-3-fluorophenyl]-2-oxo-5 oxazolidinyl]methyl]acetamide. N-[[(5S)-3-[4-(2,6-dihydro-2-methylpyrrolo[3,4-c]pyrazol-5(4H)-yl)-3-fluorophenyl]-2-oxo-5-oxazolidinyl]methyl]acetamide is described in U.S. Pat. No. 6,413,981. One issue common to many oxazolidones is low absorption rate and poor water solubility. In particular, N-[[(5S)-3-[4-(2,6-dihydro-2-methylpyrrolo[3,4-c]pyrazol-5(4H)-yl)-3-fluorophenyl]-2-oxo-5-oxazolidinyl]methyl]acetamide has been shown to have poor bioavailability in humans. A need exists to increase the bioavailability of this API.

Applicants have discovered that N-[[(5S)-3-[4-(2,6-dihydro-2-methylpyrrolo[3,4-c]pyrazol-5(4H)-yl)-3-fluorophenyl]-2-oxo-5-oxazolidinyl]methyl]acetamide can form a crystal which increases the bioavailability of this API. This discovery increases opportunities for the identification of an improved formulation suitable for efficacious use and FDA approval.

### SUMMARY OF THE INVENTION

It has now been found that a novel crystal of N-[[(5S)-3-[4-(2,6-dihydro-2-methylpyrrolo[3,4-c]pyrazol-5(4H)-yl)-3-fluorophenyl]-2-oxo-5-oxazolidinyl]methyl]acetamide can be obtained.

In one embodiment, the invention provides a Form α crystal with the chemical formula C₂₁H₂₄FN₅O₇.

In another embodiment, the invention provides a Form α crystal with the chemical formula C₂₁H₂₄FN₅O₇ and wherein said crystal comprises of N-[[(5S)-3-[4-(2,6-dihydro-2-methylpyrrolo[3,4-c]pyrazol-5(4H)-yl)-3-fluorophenyl]-2-oxo-5-oxazolidinyl]methyl]acetamide.

In another embodiment, the invention provides a Form α crystal with the chemical formula C₂₁H₂₄FN₅O₇ wherein said crystal is a co-crystal.

The invention also provides for methods of making the novel Form α crystal.

The invention also provides pharmaceutical compositions comprising this novel Form α crystal.

Compositions and methods of the invention are useful in the treatment or prevention of a variety of diseases including, among others, bacterial infections.

### DESCRIPTION OF THE FIGURES

FIG. 1 illustrates powder X-ray diffraction (PXRD) measurements of a representative Form α crystal.

FIG. 2 illustrates powder X-ray diffraction (PXRD) measurements of a representative Form α crystal.

FIG. 3 is the molecular structure of the compound N-[[(5S)-3-[4-(2,6-dihydro-2-methylpyrrolo[3,4-c]pyrazol-5(4H)-yl)-3-fluorophenyl]-2-oxo-5-oxazolidinyl]methyl]acetamide.

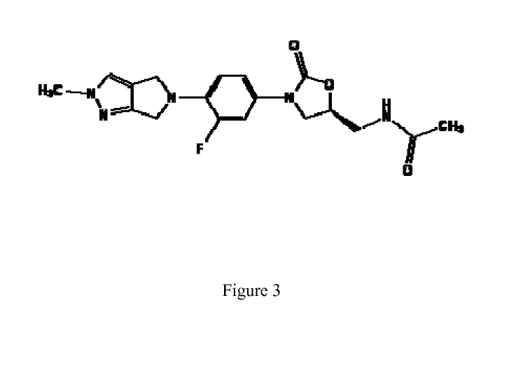

### DETAILED DESCRIPTION OF THE INVENTION

Applicants have discovered that N-[[(5S)-3-[4-(2,6-dihydro-2-methylpyrrolo[3,4-c]pyrrazol-5(4H)-yl)-3-fluorophenyl]-2-oxo-5-oxazolidinyl]methyl]acetamide can form a Form α crystal with the chemical formula C₂₁H₂₄FN₅O₇ which increases the bioavailability of this API. While Applicant's believe this Form α crystal is a co-crystal of N-[[(5S)-3-[4-(2,6-dihydro-2-methylpyrrolo[3,4-c]pyrazol-5(4H)-yl)-3-fluorophenyl]-2-oxo-5-oxazolidinyl]methyl]acetamide and malonic acid, it is possible that this Form α crystal is a malonate salt of N-[[(5S)-3-[4-(2,6-dihydro-2-methylpyrrolo[3,4-c]pyrazol-5(4H)-yl)-3-fluorophenyl]-2-oxo-5-oxazolidinyl]methyl]acetamide. Difficulties in analyzing the single crystal structure of this Form α crystal have prevented Applicant's from determining with absolute certainty whether the Form α crystal is a co-crystal or a salt. Regardless, Applicant's have isolated the Form α crystal, analyzed the Form α crystal with powder x-ray diffraction to identify the unique crystal pattern of this crystal, identified reproducible methods of making this Form α crystal, and observed an improved solubility and bioavailability with this Form α crystal.

The term “co-crystal” as used herein means a crystalline material comprised of two or more unique solids at room temperature (22 degrees C.), at least one of which is a co-crystal former. Solvates of N-[[(5S)-3-[4-(2,6-dihydro-2-methylpyrrolo[3,4-c]pyrazol-5(4H)-yl)-3-fluorophenyl]-2-oxo-5-oxazolidinyl]methyl]acetamide that do not further comprise a co-crystal former are not co-crystals according to the present invention. The co-crystals may however, include one or more solvate molecules in the crystalline lattice. An API bound to an acid or base in the form of a salt can be one unique solid, but it cannot be two unique solids by itself.

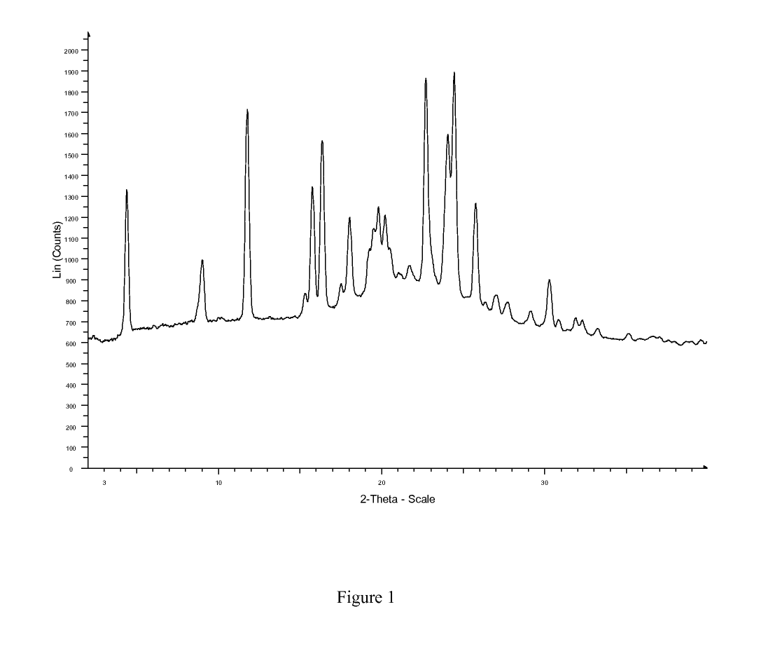

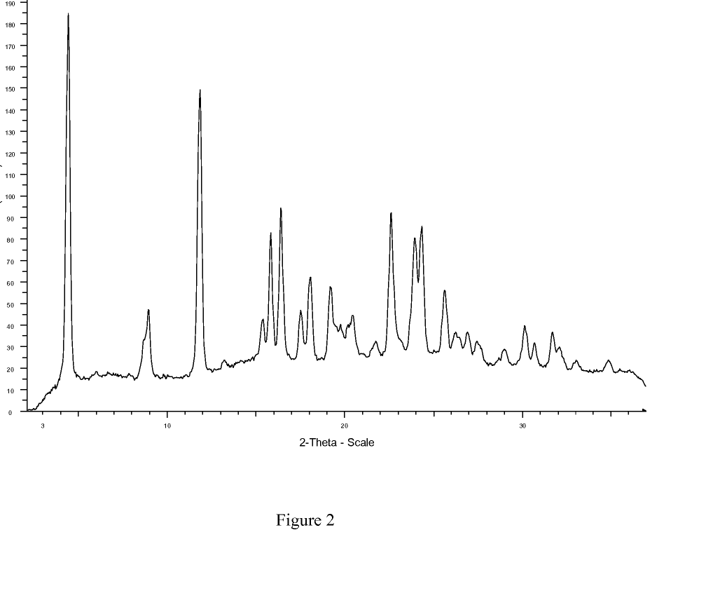

In one embodiment, the invention provides a Form α crystal with the chemical formula C₂₁H₂₄FN₅O₇. In one aspect of this invention, a Form α crystal is characterized by a powder X-ray diffraction pattern having one powder X-ray diffraction peak at about 4.3 degrees 2-theta. In another aspect of this invention, a Form α crystal is characterized by a powder X-ray diffraction pattern having powder X-ray diffraction peaks at about 4.3 and 11.7 degrees 2-theta. In one aspect of this invention, a Form α crystal is characterized by a powder X-ray diffraction pattern having powder X-ray diffraction peaks at about 4.3, 11.7, and 16.3 degrees 2-theta. In a further aspect of this invention, a Form α crystal is characterized by a powder X-ray diffraction pattern having powder X-ray diffraction peaks at about 4.3, 8.9, 11.7, 15.6, and 16.3 degrees 2-theta. In a still further aspect of this invention, a Form α crystal is characterized by a powder X-ray diffraction pattern having powder X-ray diffraction peaks at about 4.3, 8.9, 11.7, 15.6, 16.3, and 17.9 degrees 2-theta. In another aspect of this invention, a Form α crystal is characterized by a powder X-ray diffraction pattern having powder X-ray diffraction peaks at about 4.3, 8.9, 11.7, 15.6, 16.3, 17.9, 19.1, and 22.6 degrees 2-theta. In one aspect of this invention, a Form α crystal is characterized by a powder X-ray diffraction pattern that is substantially similar to the powder X-ray diffraction pattern of FIG. 1. In one aspect of this invention, a Form α crystal is characterized by a powder X-ray diffraction pattern that is substantially similar to the powder X-ray diffraction pattern of FIG. 2. In another aspect of this invention, a Form α crystal is a co-crystal.

In another embodiment, the invention provides a Form α crystal with the chemical formula C₂₁H₂₄FN₅O₇ wherein said crystal comprises N-[[(5S)-3-[4-(2,6-dihydro-2-methylpyrrolo[3,4-c]pyrazol-5(4H)-yl)-3-fluorophenyl]-2-oxo-5-oxazolidinyl]methyl]acetamide. In a further embodiment, the invention provides a Form α crystal with the chemical formula C₂₁H₂₄FN₅O₇ wherein said crystal comprises N-[[(5S)-3-[4-(2,6-dihydro-2-methylpyrrolo[3,4-c]pyrazol-5(4H)-yl)-3-fluorophenyl]-2-oxo-5-oxazolidinyl]methyl]acetamide and malonic acid. In a further embodiment, the invention provides for pharmaceutical compositions comprising a Form α crystal with the chemical formula C₂₁H₂₄FN₅O₇.

In one embodiment, the invention provides for a crystal with the chemical formula C₂₁H₂₄FN₅O₇, wherein said crystal is characterized by a powder X-ray diffraction pattern having one powder X-ray diffraction peak at about 4.3 degrees 2-theta. In another embodiment, the invention provides for a crystal with the chemical formula C₂₁H₂₄FN₅O₇, wherein said crystal is characterized by a powder X-ray diffraction pattern having powder X-ray diffraction peaks at about 4.3 and 11.7 degrees 2-theta. In a further embodiment, the invention provides for a crystal with the chemical formula C₂₁H₂₄FN₅O₇, wherein said crystal is characterized by a powder X-ray diffraction pattern having powder X-ray diffraction peaks at about 4.3, 11.7, and 16.3 degrees 2-theta. In a still further embodiment, the invention provides for a crystal with the chemical formula C₂₁H₂₄FN₅O₇, wherein said crystal is characterized by a powder X-ray diffraction pattern having powder X-ray diffraction peaks at about 4.3, 8.9, 11.7, 15.6, and 16.3 degrees 2-theta. In another embodiment, the invention provides for a crystal with the chemical formula C₂₁H₂₄FN₅O₇, wherein said crystal is characterized by a powder X-ray diffraction pattern having powder X-ray diffraction peaks at about 4.3, 8.9, 11.7, 15.6, 16.3, and 17.9 degrees 2-theta. In a further embodiment, the invention provides for a crystal with the chemical formula C₂₁H₂₄FN₅O₇, wherein said crystal is characterized by a powder X-ray diffraction pattern having powder X-ray diffraction peaks at about 4.3, 8.9, 11.7, 15.6, 16.3, 17.9, 19.1, and 22.6 degrees 2-theta.

It has been found that Form α crystal with the chemical formula C₂₁H₂₄FN₅O₇, has improved or different properties than compared to prior known forms of N-[[(5S)-3-[4-(2,6-dihydro-2-methylpyrrolo[3,4-c]pyrazol-5(4H)-yl)-3-fluorophenyl]-2-oxo-5-oxazolidinyl]methyl]acetamide. In particular, Form α has improved solubility and bioavailability.

Compositions and methods of the invention are useful in the treatment or prevention of a variety of diseases including, among others, bacterial infections, fungal infections, and infectious disease.

Assaying the solid phase for the presence of a Form α crystal may be carried out by conventional methods known in the art. For example, X-ray diffraction techniques can be used to assess the presence of co-crystals. Other techniques, used in an analogous fashion, include differential scanning calorimetry (DSC), thermogravimetric analysis (TGA), infrared spectroscopy (IR), single crystal X-ray diffraction and Raman spectroscopy. FIGS. 1 and 2 show PXRD measurements of representative Form α crystals.

In one embodiment, the invention provides for a method of making a Form α crystal with the chemical formula C₂₁H₂₄FN₅O₇ comprising the steps of cocrystallizing N-[[(5S)-3-[4-(2,6-dihydro-2-methylpyrrolo[3,4-c]pyrazol-5(4H)-yl)-3-fluorophenyl]-2-oxo-5-oxazolidinyl]methyl]acetamide with malonic acid and isolating the crystal. In another embodiment, the use of an excess (more than 1 molar equivalent for a 1:1 malonic acid) of malonic acid can be used to drive the formation of a Form α crystal. Such an excessive use of malonic acid to form a crystal can be employed in solution or when grinding N-[[(5S)-3-[4-(2,6-dihydro-2-methylpyrrolo[3,4-c]pyrazol-5(4H)-yl)-3-fluorophenyl]-2-oxo-5-oxazolidinyl]methyl]acetamide and malonic acid to cause Form α crystal formation.

The Form α crystal obtained as a result of such process steps may be readily incorporated into a pharmaceutical composition (or medicament) by conventional means. Pharmaceutical compositions and medicaments may further comprise a pharmaceutically-acceptable diluent, excipient or carrier. In one embodiment, the Form α crystal and formulations comprising N-[[(5S)-3-[4-(2,6-dihydro-2-methylpyrrolo[3,4-c]pyrazol-5(4H)-yl)-3-fluorophenyl]-2-oxo-5-oxazolidinyl]methyl]acetamide, are suitably stable for pharmaceutical use.

For preparing pharmaceutical compositions from the Form α crystal described by this invention, inert, pharmaceutically acceptable carriers can be either solid or liquid. Solid form preparations include powders, tablets, dispersible granules, capsules, cachets and suppositories. Tablets, powders, cachets and capsules can be used as solid dosage forms suitable for oral administration. Examples of pharmaceutically acceptable carriers and methods of manufacture for various compositions may be found in A. Gennaro (ed.), The Science and Practice of Pharmacy, 20.sup.th Edition, Lippincott Williams & Wilkins, Baltimore, Md., (2000).

Liquid form preparations include solutions, suspensions and emulsions. Aerosol preparations suitable for inhalation may include solutions and solids in powder form, which may be in combination with a pharmaceutically acceptable carrier, such as an inert compressed gas, e.g., nitrogen. Also included are solid form preparations that are intended to be converted, shortly before use, to liquid form preparations for either oral or parenteral administration. Such liquid forms include solutions, suspensions and emulsions.

Specific dosage and treatment regimens for any particular patient may be varied and will depend upon a variety of factors, the age, body weight, general health status, sex and diet of the patient, the time of administration, the rate of excretion, the specific drug combination, the severity and course of the symptoms being treated, the patient's disposition to the condition being treated and the judgment of the treating physician. Determination of the proper dosage regimen for a particular situation is within the skill of the art. The amount and frequency of the administration of the compositions of this invention, or the pharmaceutical compositions thereof, may be regulated according to the judgment of the attending clinician, based on the factors recited above. As a skilled artisan will appreciate, lower or higher doses than those recited above may be required.

The Crystal of the Present Invention was Analyzed Using the Following Methods.

Powder x-ray diffraction patterns were obtained using either a D/Max Rapid X-ray Diffractometer (Rigaku/MSC, The Woodlands, Tex., U.S.A.) or a Bruker D8 Discover with GADDS diffractometer (Bruker-AXS Inc., Madison, Wis., U.S.A).

The D/Max Rapid X-ray Diffractometer was equipped with a copper source (Cu/K_{α}1.5406 Å), manual x-y stage, and 0.3 mm collimator. A sample was loaded into a 0.3 mm quartz capillary tube (Charles Supper Company, Natick, Mass., U.S.A.) by sectioning off the closed end of the tube and tapping the small, open end of the capillary tube into a bed of the powdered sample or into the sediment of a slurried sample. The loaded capillary tube was mounted in a holder that was placed and fitted into the x-y stage. A diffractogram was acquired using control software (RINT Rapid Control Software, Rigaku Rapid/XRD, version 1.0.0 (©1999 Rigaku Co.)) under ambient conditions at a power setting of 46 kV at 40 mA in transmission mode, while oscillating about the omega-axis from 0-5 degrees at 1 degree/second, and spinning about the phi-axis over 360 degrees at 2 degrees/second. The exposure time was 15 minutes unless otherwise specified.

The diffractogram obtained was integrated of 2-theta from 2-40 degrees and chi (1 segment) from 0-36 degrees at a step size of 0.02 degrees using the cyllnt utility in the RINT Rapid display software (RINT Rapid display software, version 1.18 (Rigaku/MSC)) provided by Rigaku with the instrument. The dark counts value was set to 8 as per the system calibration by Rigaku. No normalization or omega, chi, or phi offsets were used for the integration.

The Bruker D8 Discover with GADDS Diffractometer was equipped with a copper source (Cu/K_{α}1.5406 Å), computer controlled x-y-z stage, a 0.5 mm collimator and a Hi-Star area detector. Samples were loaded into a proprietary sample holder by tapping the sample holder into a powder bed and arraying the holders into a 96 position block. The block was then loaded onto the x-y-z stage and the sample positions were entered into the software. A diffractogram was acquired using control software (GADDS—General Area Detector Diffraction System, (Bruker, version 4.1.14 (©1997-2003 Bruker-AXS.)) under ambient conditions at a power setting of 46 kV at 40 mA in reflectance mode. The exposure time was 5 minutes unless otherwise specified.

The diffractogram obtained was integrated of 2-theta from 2-40 degrees and chi (1 segment) from 0-36 degrees at a step size of 0.02 degrees using the GADDS software.

The relative intensity of peaks in a diffractogram is not necessarily a limitation of the PXRD pattern because peak intensity can vary from sample to sample, e.g., due to crystalline impurities. Further, the angles of each peak can vary by about +/−0.2 degrees, or by about +/−0.1. The entire pattern or most of the pattern peaks may also shift by about +/−0.1 degrees to about +/−0.2 degrees due to differences in calibration, settings, and other variations from instrument to instrument and from operator to operator. All reported PXRD peaks in the Figures, Examples, and elsewhere herein are reported with an error of about ±0.2 degrees 2-theta. Unless otherwise noted, all diffractograms are obtained at about room temperature (about 24 degrees C. to about 25 degrees C.).

For PXRD data herein, including Tables and Figures, each composition of the present invention may be characterized by any one, any two, any three, any four, any five, any six, any seven, or any eight or more of the 2 theta angle peaks.

The following specific examples illustrate the present invention in more detail. They are, however, not intended to limit its scope in any manner.

### EXAMPLES

### Example 1

### Cocrystalization of a Form α Crystal

N-[[(5S)-3-[4-(2,6-dihydro-2-methylpyrrolo[3,4-c]pyrazol-5(4H)-yl)-3-fluorophenyl]-2-oxo-5-oxazolidinyl]methyl]acetamide (˜10 mg, 0.027 mmol) was ground in a ball mill with malonic acid (˜6 mg, 0.054 mmol) without solvent or with acetone, acetonitrile, 1,4-dioxane, methanol, or nitromethane (10 □L) for 10 min. The resulting solids were analyzed by powder X-ray diffraction.

### Example 2

### Cocrystalization of a Form α Crystal

N-[[(5S)-3-[4-(2,6-dihydro-2-methylpyrrolo[3,4-c]pyrazol-5(4H)-yl)-3-fluorophenyl]-2-oxo-5-oxazolidinyl]methyl]acetamide (100 mg, 0.27 mmol) was added to malonic acid (29 mg, 0.28 mmol) and anisole (6.0 g). The contents were stirred at room temperature overnight. The resulting solid was analyzed by powder X-ray diffraction.

### Example 3

### Cocrystalization of a Form α Crystal

N-[[(5S)-3-[4-(2,6-dihydro-2-methylpyrrolo[3,4-c]pyrazol-5(4H)-yl)-3-fluorophenyl]-2-oxo-5-oxazolidinyl]methyl]acetamide (99 mg, 0.27 mmol) was added to malonic acid (29 mg, 0.28 mmol) and toluene (6.0 g). The contents were stirred at room temperature overnight. The resulting solid was analyzed by powder X-ray diffraction.

### Example 4

### Cocrystalization of a Form α Crystal

N-[[(5S)-3-[4-(2,6-dihydro-2-methylpyrrolo[3,4-c]pyrazol-5(4H)-yl)-3-fluorophenyl]-2-oxo-5-oxazolidinyl]methyl]acetamide (14.2 g, 0.038 mol) was added to malonic acid (4.5 g, 0.043 mol) and anisole (851 g). The contents were stirred at 30° C. for 7 h. The solid was filtered using a Buchner funnel and dried in a vacuum oven for 4 days. The resulting solid was analyzed by powder X-ray diffraction.

### Example 5

### Cocrystalization of a Form α Crystal

N-[[(5S)-3-[4-(2,6-dihydro-2-methylpyrrolo[3,4-c]pyrazol-5(4H)-yl)-3-fluorophenyl]-2-oxo-5-oxazolidinyl]methyl]acetamide (101 mg, 0.270 mmol) was added to malonic acid (42 mg, 0.403 mmol), isopropyl acetate (3 mL) and hexanes (3 mL). The contents were heated at 30° C. while stirring for 4 h. The contents were then left stirring at room temperature for 3 days. The solid was filtered using a Buchner funnel and washed with hexanes. The solid was dried in a vacuum oven for 3 days. The resulting solid was analyzed by powder X-ray diffraction.

### Example 6

### Cocrystalization of a Form α Crystal

N-[[(5S)-3-[4-(2,6-dihydro-2-methylpyrrolo[3,4-c]pyrazol-5(4H)-yl)-3-fluorophenyl]-2-oxo-5-oxazolidinyl]methyl]acetamide (13.3, 0.036 mol) was added to malonic acid (5.6 g, 0.054 mol), isopropyl acetate (150 g) and hexanes (150 g). Seed Form a crystal was also added. The contents were stirred at room temperature for 5 days. The solid was filtered using a Buchner funnel and dried in a vacuum oven for 3 days. The resulting solid was analyzed by powder X-ray diffraction.

### Example 7

### Cocrystalization of a Form α Crystal

341 g of DMSO was heated to 80° C. Then, 20 g of N-[[(5S)-3-[4-(2,6-dihydro-2-methylpyrrolo[3,4-c]pyrazol-5(4H)-yl)-3-fluorophenyl]-2-oxo-5-oxazolidinyl]methyl]acetamide was dissolved and stirred. 308 g malonic acid was added to the earlier contents and stirred. 40 g of deionized water was added to the contents and stirred. The contents were cooled down to 0° C. and stirred. The contents were filtered in centrifuge filter tubes to remove solvent. The solids were washed with isopropyl acetate (approx 10 ml per g solid). The solids were dried under vacuum at room temperature overnight. The resulting solid was analyzed by powder X-ray diffraction.

### Example 8

### Cocrystalization of a Form α Crystal

63.2 g of malonic acid were added to 1120 g isopropyl acetate at room temperature and stirred. 50.0 g of N-[[(5S)-3-[4-(2,6-dihydro-2-methylpyrrolo[3,4-c]pyrazol-5(4H)-yl)-3-fluorophenyl]-2-oxo-5-oxazolidinyl]methyl]acetamide was added to these earlier contents and stirred for 18 hours. The contents were filtered and washed with isopropyl acetate (approx 8 ml per g solid). The solids were dried under nitrogen at 40° C. overnight. The resulting solid was analyzed by powder X-ray diffraction.

1. A Form α crystal with the chemical formula C₂₁H₂₄FN₅O₇.

2. The crystal of claim 1, wherein said crystal is characterized by a powder X-ray diffraction pattern having one powder X-ray diffraction peaks at about 4.3 and 11.7 degrees 2-theta.

3. The crystal of claim 1, wherein said crystal is characterized by a powder X-ray diffraction pattern having powder X-ray diffraction peaks at about 4.3, 11.7, and 16.3 degrees 2-theta.

4. The crystal of claim 1, wherein said crystal is characterized by a powder X-ray diffraction pattern having powder X-ray diffraction peaks at about 4.3, 8.9, 11.7, 15.6, and 16.3 degrees 2-theta.

5. The crystal of claim 1, wherein said crystal is characterized by a powder X-ray diffraction pattern having powder X-ray diffraction peaks at about 4.3, 8.9, 11.7, 15.6, 16.3, and 17.9 degrees 2-theta.

6. The crystal of claim 1, wherein said crystal is characterized by a powder X-ray diffraction pattern having powder X-ray diffraction peaks at about 4.3, 8.9, 11.7, 15.6, 16.3, 17.9, 19.1, and 22.6 degrees 2-theta.

7. The crystal of claim 1, wherein said crystal is a co-crystal.

8. A crystal with the chemical formula C₂₁H₂₄FN₅O₇, wherein said crystal is characterized by a powder X-ray diffraction pattern having one powder X-ray diffraction peak at about 4.3 degrees 2-theta.

9. The crystal of claim 8, wherein said crystal is characterized by a powder X-ray diffraction pattern having powder X-ray diffraction peaks at about 4.3 and 11.7 degrees 2-theta.

10. The crystal of claim 8, wherein said crystal is characterized by a powder X-ray diffraction pattern having powder X-ray diffraction peaks at about 4.3, 11.7, and 16.3 degrees 2-theta.

11. The crystal of claim 8, wherein said crystal is characterized by a powder X-ray diffraction pattern having powder X-ray diffraction peaks at about 4.3, 8.9, 11.7, 15.6, and 16.3 degrees 2-theta.

12. The crystal of claim 8, wherein said crystal is characterized by a powder X-ray diffraction pattern having powder X-ray diffraction peaks at about 4.3, 8.9, 11.7, 15.6, 16.3, and 17.9 degrees 2-theta.

13. The crystal of claim 8, wherein said crystal is characterized by a powder X-ray diffraction pattern having powder X-ray diffraction peaks at about 4.3, 8.9, 11.7, 15.6, 16.3, 17.9, 19.1, and 22.6 degrees 2-theta.

14. A crystal with the chemical formula C₂₁H₂₄FN₅O₇, wherein said crystal comprises N-[[(5S)-3-[4-(2,6-dihydro-2-methylpyrrolo[3,4-c]pyrazol-5(4H)-yl)-3-fluorophenyl]-2-oxo-5-oxazolidinyl]methyl]acetamide.

15. The crystal of claim 14, wherein said crystal comprises malonic acid.

16. The crystal of claim 14, wherein said crystal is characterized by a powder X-ray diffraction pattern having powder X-ray diffraction peaks at about 4.3 and 11.7 degrees 2-theta.

17. The crystal of claim 14, wherein said crystal is characterized by a powder X-ray diffraction pattern having powder X-ray diffraction peaks at about 4.3, 11.7, and 16.3 degrees 2-theta.

18. The crystal of claim 14, wherein said crystal is characterized by a powder X-ray diffraction pattern having powder X-ray diffraction peaks at about 4.3, 8.9, 11.7, 15.6, and 16.3 degrees 2-theta.

19. The crystal of claim 14, wherein said crystal is characterized by a powder X-ray diffraction pattern having powder X-ray diffraction peaks at about 4.3, 8.9, 11.7, 15.6, 16.3, and 17.9 degrees 2-theta.

20. The crystal of claim 14, wherein said crystal is characterized by a powder X-ray diffraction pattern having powder X-ray diffraction peaks at about 4.3, 8.9, 11.7, 15.6, 16.3, 17.9, 19.1, and 22.6 degrees 2-theta.

21. A method of making a crystal with the chemical formula C₂₁H₂₄FN₅O₇, comprising the steps of cocrystallizing N-[[(5S)-3-[4-(2,6-dihydro-2-methylpyrrolo[3,4-c]pyrazol-5(4H)-yl)-3-fluorophenyl]-2-oxo-5-oxazolidinyl]methyl]acetamide with malonic acid and isolating the crystal.

22. A crystal obtained by the cocrystallization of N-[[(5S)-3-[4-(2,6-dihydro-2-methylpyrrolo[3,4-c]pyrazol-5(4H)-yl)-3-fluorophenyl]-2-oxo-5-oxazolidinyl]methyl]acetamide with malonic acid.
